(19) United States
(12) Patent Application Publication
(10) Pub. No.: US 2007/0297991 A1
(43) Pub. Date: Dec. 27, 2007

(54) NEURAL CONDUIT AGENT DISSEMINATION FOR SMOKING CESSATION AND OTHER APPLICATIONS

(75) Inventor: Gholam A. Peyman, Tucson, AZ (US)

Correspondence Address: THOMPSON HINE L.L.P.;Intellectual Property Group, P.O. BOX 8801, DAYTON, OH, 45401-8801, US

(73) Assignee: MINU, L.L.C., Pittsboro, NC (US)

(21) Appl. No.: 11/530,514

(22) Filed: Sep. 11, 2006

Related U.S. Application Data
(60) Provisional application No. 60/815,940, filed on Jun. 23, 2006.

(51) Int. Cl. A61K 48/00; A61K 38/17; A61K 31/4439; A61N 1/30; A61K 9/14

(52) U.S. Cl. 424/46; 514/12; 514/44; 604/20; 514/343

(57) ABSTRACT

A method for disseminating nicotine or another agent using a neural conduit for direct transport from a peripheral nervous system site of administration to a central nervous system site of operation. In one embodiment, the agent may be provided to a sensory organ, such as the nose, for dissemination along a nasal neural conduit to the brain.

### RELATED APPLICATION

This application claims priority to provisional Patent Application Ser. No. 60/815,940 filed on Jun. 23, 2006, the disclosure of which is expressly incorporated by reference herein in its entirety.

### FIELD OF THE INVENTION

A method and system using neural conduits to disseminate agents, e.g., to assist in smoking cessation and reduced nicotine craving and other applications.

### BACKGROUND

Nicotine-related disorders include, but are not limited to, nicotine dependence, nicotine withdrawal, and nicotine-related disorders not otherwise specified (NOS). Various compositions and methods have been used to reduces the craving for nicotine and thus for smoking cessation. For example, nicotine lozenges provide periodic doses of nicotine when an individual who is attempting to quit smoking swallows the dissolving lozenge. Nicotine transdermal systems, commonly referred to as patches, provide a low consistent nicotine blood level while bypassing the metabolic gut and liver first pass effect. Nicotine vapor devices deliver a periodic nicotine aerosol and are administered similar to inhalers used to supply bronchial asthma medications. Several varieties of smokeless cigarettes are available that provide nicotine without the tar and other carcinogenic products of tobacco. Other smokeless cigarettes do not contain nicotine but instead contain natural herbal ingredients and/or food-grade flavorings.

Smoking a cigarette delivers nicotine to the lungs, where it is rapidly absorbed through the arteries and delivered to the brain. In the brain, nicotine interacts with nicotinic cholinergic receptors to induce neurotransmitter release and produce an immediate reward—the “rush” associated with a rapid rise in nicotine blood lever that a smoker experiences. A persistent stimulus associated with a high blood nicotine level is also produced. As such, the dopaminergic reward system is activated, which eventually results in nicotine dependency. Complex behavioral and social aspects of smoking, e.g., the hand-to-mouth ritual, etc., are also habit-forming. Intravenous administration of various compounds, such as (−)-1-(3,4-dichlorophenyl)-3-azabicyclo[3.1.0]hexane, has ben described as inhibiting dopamine reuptake in the treatment of nicotine related disorders.

Other methods are desirable.

### DETAILED DESCRIPTION

A method and system to disseminate agents. In one embodiment, the method and system assist in smoking cessation by reducing an individual's nicotine craving.

Nicotine or another biocompatible agent, as subsequently described and collected termed “agent”, is disseminated through a neural conduit by administering the agent to a peripheral nervous system site in decreasing doses over time, such that the agent is transported substantially by the neural conduit, and substantially free of a vascular conduit, to at least one cholinergic receptor in the central nervous system, thus decreasing the amount and/or duration of the agent that is needed for reduced nicotine cravings by use of the neural conduit.

A neural pathway provides the agent to a proximal and/or distal neural site. The agent uses neural, rather than vascular conduits to reach its targets in the central and/or peripheral nervous system. Thus, the agent is neuronally transported, providing shorter and more direct access to its nervous system target site. This is contrasted with vascular transport, which is a longer and less direct route with the dilution effects of the agent in blood. Over time and with decreasing doses, the method reduces nicotine addiction and reduces or prevents undesirable withdrawal symptoms.

Agent, as used herein, is at least one biocompatible compound effective to reduce nicotine addiction and/or the effects of nicotine. These include nicotine, nicotine analogs and agonists as disclosed in U.S. Pat. No. 6,277,855 which is expressly incorporated by reference herein, trans-metanicotine and its analogs, epibatidine and its analogs, pyridol derivatives, piperidine alkaloids such as lobeline and its analogs, certain para-alkylthiophenol derivatives, imidaceliprid and its analogs, and any other compounds and their derivatives and/or analogues, or antagonists and their derivatives and/or analogues, which are used for treating nicotine addiction, known to one skilled in the art. As non-limiting examples, (−)-1-(3,4-dichlorophenyl)-3-azabicyclo[3.1.0]hexane inhibits dopamine reuptake in treating nicotine related disorders (U.S. Pat. No. 7,041,835) and may be disseminated using the disclosed system and method. Heteroaryl diazabicycloalkane derivatives and enantiomers, and their pharmaceutically acceptable salts, are ligands at cholinergic receptors, specifically the nicotinic acetyl choline receptors (U.S. Patent Application Publication 2002/0037893); (+)-2S,3S)-2-(3-chlorophenyl)-3,5,5-trimethyl-2-morpholinol and its pharmaceutically acceptable salts and solvates is used to treat various disorders including addiction to tobacco products (U.S. Pat. No. 6,998,400). These compounds may be used as agents in the disclosed method. The above references are expressly incorporated by reference herein in their entirety.

Other non-limiting examples of agent include anti-nicotine and smoking cessation agents such as the following compounds and their salts and analogs: clonidine, bupropion, ibogaine, transmetanicotine, epibatidine, lobeline, pyridol derivatives, para-alkylthiophenol derivatives, imidacloprid, topiramate, and vigabatrin (gamma vinyl gamma amino butyric acid).

An effective dose of agent to reduce or inhibit cholinergic receptor number of function, and to reduce or inhibit dopamine reuptake, is administered to an individual. Upon administration, the agent is disseminated substantially directly via at least one neural conduit to a proximal and/or distal neural site. This occurs in the substantial absence of transport of the agent through a vascular conduit (i.e., a blood vessel). In one embodiment the agent is nicotine. In another embodiment the agent is effective to provide the described receptor and/or uptake effect. The method and system facilitates the individual's temporal weaning from nicotine. In one embodiment, this occurs to a point whereby the individual no longer has a desire to smoke and is able to quit smoking.

In one embodiment, an agent is provided at a site in the peripheral nervous system (PNS), which includes sensory organs such as the nose and tongue, to provide agent to the central nervous system (CNS). In another embodiment an agent is provided at a site in the central nervous system for diffuse dissemination and/or channeled dissemination of agent to the peripheral nervous system along neural conduits. In another embodiment, a neural conduit provides an agent to one or more specific areas in the central nervous system and/or peripheral nervous system.

It is known that silicone oil administered intravitreally to an individual for retinal tamponade migrated along the intracranial portion of the optic nerve into the brain. Retrograde transport to the brain from each of the optic nerve and the retina has been reported. It is known that an opioid may be topically administered intranasally and absorbed through the nasal mucosa to relive migraines, as disclosed in U.S. Pat. No. 5,856,807 which is expressly incorporated by reference herein in its entirety. Efficacy of topical intranasal administration may involve the sphenopalatine ganglion (SPG) of the trigeminal system, located immediately posterior to and immediately above the posterior tip of the middle turbinate behind the nasal mucosa. SPG has sensor, parasympathetic, and sympathetic nerve supplies. It is the major source of parasympathetic innervation to brain vasculature, and its stimulation increases blood brain barrier permeability (Yarnitsky et al., Brain Res. 1018: 236 (2004)). The method uses such a central and/or peripheral neural conduit for agent administration, delivery, and dissemination.

The method is capable of providing a titrated dose of the agent to temporally attenuate and ultimately alleviate the effects of nicotine in an individual. In one embodiment, the method is self-administered, either partially or totally. For example, an individual may regulate time but not dose, or dose but not time, etc. In another embodiment, the method is practioner-regulated. In another embodiment, the method may be used in conjunction with other methods (e.g., behavior therapy, smokeless cigarettes, etc.).

In one embodiment, the method provides an agent that substitutes for nicotine to replace, decrease, or alleviate the side-effects that result from smoking tobacco, for example, caffeine, S-adenosyl methionine (SAM) and others as described in U.S. Patent Application Publication No. 2005/0241658 which is expressly incorporated by reference herein. The method may also aid in nicotine withdrawal symptoms that are associated with the physiological and/or behavioral effects of nicotine use or its withdrawal.

In one embodiment, the agent is administered at a first site in the peripheral nervous system, for example, the sensory system, for action in a second site in the sensory or other peripheral nervous system site, and/or for action in the central nervous system. In another embodiment, the agent is administered at a site within the central nervous system and transported for action to other sites in the central nervous

As used herein and as generally recognized by one skilled in the art, the central nervous system encompasses the brain and spinal cord. Access to specific regions within the central nervous system may be limited by neuroanatomy and/or neurophysiology. As one example, a neural conduit may be used to provide nicotinic acetylcholinergic receptor agonists as agents to inhibit cholinergic receptor function and/or inhibit dopamine reuptake into cells.

As used herein and as generally recognized by one skilled in the art, the peripheral nervous system encompasses sensory afferent nerves and motor efferent nerves. Motor efferent nerves further include the somatic nervous system controlling voluntary skeletal muscles, and the autonomic nervous system. The autonomic nervous system has a parasympathetic component to maintain homeostasis, and a sympathetic component to permit a fight or flight response. The inventive method may use any or all of these neural conduits.

In the central nervous system, a unique membranous barrier tightly segregates the brain from the circulating blood. The barrier function is due to the capillaries in the central nervous system that are structurally different from capillaries in other tissues; these structural differences result in a permeability barrier (blood brain barrier, BBB) between blood maintained within these capillaries and the extracellular fluid in brain. In vertebrates, these brain and spinal cord capillaries lack the small pores or fenestrations that allow rapid movement of agents from the circulation into other organs; instead, they are lined with a layer of special endothelial cells that are sealed with tight junctions. These capillaries make up about 95% of the total surface area of the BBB, and represent the principal route through which chemicals enter the brain. The capillaries have smaller diameters and thinner walls than capillaries in other organs. Because they essentially lack intercellular clefts, pinocytosis functions, and fenestrae, any exchange into or out of these capillaries must pass trans-cellularly (across cells). Therefore, only lipid-soluble agents that can freely diffuse through the capillary endothelial membrane passively cross the BBB.

In addition to its structural barrier aspect, the BBB also has an enzymatic aspect. Agents crossing the capillary endothelial cell membrane are exposed to mitochondrial enzymes that recognize and rapidly degrade most pepticles, including naturally occurring neuropeptides.

Segregation of agents from the central nervous system, i.e., outside the BBB, is further reinforced by a high concentration of P-glycoprotein (Pgp) active-drug-efflux-transporter proteins in the capillary endothelial cell luminal membrane. These efflux transporter proteins actively remove a broad range of agents from the cytoplasm of endothelial cells before the agents can cross into the brain parenchyma.

Segregatior mechanisms such as these render the brain essentially inaccessible to many agents, including lipid-insoluble (i.e., hydrophilic) compounds, for example, polar molecules and small ions. As a consequence, the therapeutic value of otherwise promising agents is diminished, and cerebral diseases are rendered refractory to therapeutic interventions.

Neurons, specialized cells within the central and peripheral nervous systems that conduct electrochemical impulses termed action potentials, are composed of a cell body that contains the nucleus and other organelles, an axon, and dendrites. An axon is a cytoplasmic extension of the cell body and is controlled by the cell body. Axons can be of considerable length and require a steady transport of materials (e.g., vesicles, mitochondria) from the cell body along its entire length. Transport is driven by proteins, termed kinesins and dyneins that move along microtubules in the axon. Dendrites are the site of origin of nerve impulses, which are then conducted along the axon.

Neuronal transport is the general term for movement of large molecules within cell bodies. Molecules may be moved within a cell (intraneuronal transport) and between cells (interneuronal transport). Neurons efficiently communicate and transport agents to and from the cell body to the axons and dendrites. Both slow and fast transport mechanisms are used. Proteins, such as cytoskeletal structural proteins and many enzymes, are carried by slow axonal transport. Agents required at synapses between nerves are carried by more rapid axonal transport. Different protein populations are transported along axons and dendrites, so the proteins are likely sorted in the cell body into separate and distinctive types of transport vesicles. Chemical communication occurs in both directions. Retrograde transport provides larger materials back from the axons and dendrites to the cell body and is a relatively slower transport process. Anterograde transport provides smaller materials from the cell body to the termini and is a relatively faster process.

Viral-mediated neuronal transport mechanisms have been used in an attempt to target agents into the brain using retrograde transport. Some viruses have evolved an ability to use nerve transport to gain access to the nervous system, which otherwise is well protected against foreign invasion. These neurotrophic viruses, such as polio virus and herpes virus, are typically very specific in the areas of the nervous system that they attack and effect. An adeno-associated viral vector was used to target delivery of a neuroprotective gene to defined neuronal populations. Viral delivery to axon termini in the hippocampus and striatum resulted in viral internalization, retrograde transport, and transgene expression in specific projection neurons in the entorhinal cortex and substantia nigra. Using viral vectors in the nervous system, however, raises practical and safety issues.

Certain carbohydrate-binding proteins such as lectins have been used to transport agents to neurons or other target cells and within neurons via neuronal transport, for putative treatment of neurologically related conditions. Specific lectin compositions are known (e.g., U.S. Patent Application Publication No. 2005/0027119) and include a non-toxic lectin transport entity operable linked to a therapeutic agent so that the agent is capable of being transported to a target. A method for treating a neurological condition includes administering the therapeutic agent and lectin to a patient needing treatment for a neurological condition, with the therapeutic agent operably linked to a non-toxic lectin so that the therapeutic agent is capable of being transported to a target associated with the neurological condition.

Other mechanisms can be used to provide agents using neural conduits to or from the central nervous system. In one embodiment, the method utilizes a localized site in the peripheral nervous system to disseminate the agent at diffuse sites in the central nervous system. The ventricles of the brain and diffusive distribution through the cerebrospinal fluid provide an agent to the brain and/or spinal cord (central nervous system).

In an embodiment where agent is administered at one or more peripheral nervous system sites, e.g., the eye, nasal mucosa, etc., the agent interacts with microtubules in the axons by which the agent is transported. The microtubules run the length of the axon, providing a system of tracks.

Neural transport encompasses intraneuronal transport, interneuronal transport, transsynaptic transport, transport from the peripheral nervous system to the central nervous system, transport from one site in the peripheral nervous system to another site (proximal or distal) in the peripheral nervous system, transport from the central nervous system to the peripheral nervous system, and/or transport from one site in the central nervous system to another site (proximal or distal) in the central nervous system. Intraneuronal transport encompasses agent movement within the neuron following its introduction into the neuron. It includes transport from axonal nerve terminals to the cell body (retrograde transport), transport from dendritic nerve terminals to the cell body (retrograde transport), transport from dendritic nerve terminals to axonal nerve terminals, transport from axonal nerve terminals to dendritic nerve terminals, and transport to axonal and dendritic nerve terminals from the cell body (anterograde transport). Interneuronal transport encompasses transsynaptic transport whereby agent moves from one neuron to another across the synaptic space. Following introduction of the agent at a first neuron, the agent is transported to second, third, and/or higher order neurons, which are in turn synaptically connected to subsequent neurons. The mechanism of transsynaptic transport includes, but is not limited to, exocytosis from the primary neuron followed by endocytosis by the secondary neuron. The exocytotic and endocytotic events may include vesicle and/or granule mediated release and uptake, with the agent incorporated within a membrane bound organelle.

In one embodiment, interneuronal transport is used to target agents into the central nervous system, to include the brain and the spinal cord, through introduction into the peripheral nervous system, to include the motor and sensory systems. In one embodiment, agent may diffuse through the perineurium that is the sheath of connective tissue enclosing a bundle of nerve fibers. For example, the optic nerve perineurium may be a conduit between the eye and the central nervous system.

In one embodiment, the substance is provided to the site in a pro-entraining form, and then forms the substance at the site where it is provided, for example, providing thrombin and fibrinogen, which then forms a fibrin entraining network in situ.

This embodiment enhances controlled localization, positioning, or placement of, for example, an agent at an anatomical and/or physiological site where it is desirable to locate the agent. In one embodiment, the method localizes an agent that enhances smoking cessation, and/or reduces nicotine addiction symptoms or effects.

In one embodiment, the agent may be administered in a particle formulation. In one embodiment, the particle size is in the range of about 25 nm to about 200 nm. In one embodiment, the particle size is about 150 nm. Particles include microspheres, nanospheres, liposomes, microcapsules, nanocapsules, etc.

In one embodiment, the agent is entirely or partially contained in a microsphere, and the microsphere is transported using the microtubule system. In one embodiment, the microsphere is biodegradable and releases the agent as it degrades. In another embodiment, the microsphere is fabricated with controlled release properties (e.g., slow release, sustained release, delayed release, etc.). the microsphere may have moieties conjugated to its outer surface to facilitate transport intraneuronally and interneuronally, as previously described.

In one embodiment, the inventive method enhances agent containment by providing a material or substance by which or in which the agent is contained or retained along a neural conduit. The material or substance is any biocompatible material that will retain, entrain, encapsulate, and/or contain the agent as it is transported. In one embodiment, the method provides controlled release of the contained agent. The agent may be provided with a substance that will not significantly spread or migrate after injection. The agent may be mixed into the substance, or may be provided essentially simultaneously with the substance. In one embodiment, the substance is one or more of a natural and/or synthetic semisolid, gel, hydrogel, colloid, reticular network, matrix, etc. In one embodiment, the substance forms in situ. In one embodiment, the substance is a hydrogel liquid below body temperature, but gels to form a shape-retaining semisolid hydrogel at or near body temperature. In one embodiment, the substance is polyethylene glycol (PEG). In one embodiment, the substance is one or more of polyanhydrides; polyorthoesters; polylactic acid and polyglycolic acid and copolymers thereof; collagen; protein polymers; polymers, copolymers, and derivatives of polyester, polyolefin, polyurethane, polystyrene, polyethylene glycol/polyethylene oxide, polyvinylalcohol, etc.

In one embodiment, the substance is a combination of fibrinogen and thrombin that, when mixed, forms a reticular or network structure (e.g., a fibrin network). As known to one skilled in the art, the structure of fibrin may be altered by varying the concentration of thrombin mixed with fibrinogen. Relatively lower thrombin concentrations produce relatively thicker fibrin fibrils with a larger pore size, slower setting rate, and slower degradation rate. Thus, the substance may be altered to contain a vector for a desired duration and with a desired durability, delivery rate, degradation rate, geometry, etc., as known to one skilled in the art. The vector(s) may be mixed with either fibrinogen and/or thrombin and injected together to create a vector entrapped inside the mesh of fibrin. Containment of the vectors at a desired site enhances control of the gene product, for example, by reduced spreading immediately after administration (e.g., injection, implantation, etc.).

The method may be used for delivering an agent only at a defined physiological or anatomical location, e.g., at or in a defined area or tissue. The method may also be used for modifying release over time to provide sustained or controlled release. An extended release formulation is also termed a controlled release formulation, formulated so that the release of the agent occurs in an extended or controlled fashion in contrast to, for example, a bolus introduction. An alternative embodiment is a delayed release formulation, formulated to minimize or prevent the agent located at a site other than a desired site. Both extended release forms and delayed release forms are termed modified release forms.

In one embodiment, the agent may be in a microencapsulated form. Any of the above-described particles may be microencapsulated. In one embodiment, an agent is contained in particles produced through nanotechnology. Examples include soft absorbent nanoparticles, and nanoparticles with rigid shells. Other examples may be a polyvinyl alcohol hydrogel with a diameter in the range of about 500 nm to about 750 nm; a poly-N-isopropylacrylamide hydrogen (50 nm to 1 μm); a copolymer of poly(ethylene oxide)-poly(L-lactic acid); or poly(L-lactic acid) coated with poly(ethylene oxide). In another embodiment, the entrainment substance is a reservoir or depot for the vectors within an anatomical or physiological site.

In one embodiment, a peptide agent may be conjugated with one or more moieties that assist in axon transport and thus facilitate the endogenous transport mechanisms. Moieties that are capable of neuronal transport include, but are not limited to, those that interact with the endogenous transport machinery including dynein, kinesin, and myosin. As one example, small consensus binding sequences of 10-25 amino acids from the binding partners of the dynein light chains Tctex-1 and 8 (LC8) facilitate interaction between the agent and dynein. A peptide that is based on either Tctex-1 or LC8 binding peptide sequences can link a peptide agent to dynein and thus facilitate neuronal transport. As another example, the nuclear localization sequence (NLS) from the protein importin, also known as karyopherin, is another moiety that can that can link a peptide agent to dynein and thus facilitate neuronal transport. Transport-facilitating moieties can also include those that interact with endogenous agents to endogenous transport.

Interneuronal transport of a peptide agent can also be facilitated by conjugating, using methods known to one skilled in the art, the peptide agent to a moiety that is capable of transsynaptic transport. These moieties include, but are not limited to, cholera toxin B subunit (CTB), tetanus toxin C fragment (TTC), lectins (carbohydrate binding moieties such as wheat germ agglutinin (WGA), neurotrophins such as nerve growth factor; NGF), brain-derived neurotrophic factor (BDNF), and the neurotrophins NT-3, NT-4/5 and NT-6), and neurotrophic viruses that include α-herpes viruses such as herpes simplex type 1, pseudorabies viruses, and rhabdoviruses. For example, the peptide agent can be operationally coupled to the tetanus toxin C fragment. Alternatively, the genetic material encoding the peptide agent can be incorporated within a virus capable of transynaptic transport, such as a pseudorabies virus.

In one embodiment, a small molecule agent may be conjugated to an organic mimetic that facilitates agent transport. As an example, a mimetic modeled after the NLS of the HIV-1 matrix protein may be used, as known to one skilled in the art.

In one embodiment, a dye that diffuses along the length of the axon may allow visualization of an axon or dendrite. A high concentration of dye is directly injected into the neuronal process through a micropipette.

Agent may be introduced via invasive, minimally invasive, or non-invasive routes. Without being bound by a specific mechanism, a topical route of administration, even when coupled with a facilitating mechanism or compound, may be less desirable than a more invasive route of administration due to, for example, neuron proximity or other factors. In one embodiment, the agent is introduced into the eye by an ocular route. Examples include, but are not limited to, topical administration (e.g., liquid drops, ointment, cream). If injected, e.g., subconjunctival, retrobulbar, subretinal, intraretinal, or intravitreal, the agent may be injected directly into and/or adjacent a nerve root, nerve fiber or bundle such that it is neuronally disseminated, e.g., into or adjacent the sphenopalatine ganglion. The agent may be injected into dorsal root ganglion for transport of agent to the somatosensory system. The agent may be injected into regions of the optic nerve or retina for transport to the visual system, to other components of the peripheral nerve system, or to the central nervous system.

In one embodiment, the agent is introduced at one or more sites in the peripheral nervous system that are used as acupuncture sites. Administration methods may include, but are not limited to, subcutaneous injection, topical administration, transdermal administration, any of which may be either non-facilitated or facilitated. Facilitated administration includes the use of electrical current (e.g., iontophoresis), thermal energy (e.g., heat), ultrasound energy, radiant energy (e.g., laser, infrared, near-infrared, mid-infrared), etc. to disseminate agent to the desired site, at the desired interval, etc.

Most acupuncturists use traditionally identified points mapped to 14 major meridian lines, one meridian for each of the 12 inner organs, one meridian along the spine (called the governing vessel), and another along the midline of the abdomen (called the conception vessel). However, the number of points identified by acupunturists have vastly increased. There are extra meridians (some of them outlined in ancient times, others modern) with their own sets of points, there are special points (off meridians), and there are complete mappings of body structures and functions by points along the outer ears, on the nose, in the scalp, on the hands, on the feet, and at the wrists and ankles.

In one embodiment, facilitated administration of agent may be used. Examples of facilitated administration include application of electrical current, ultrasound energy, radiant energy, thermal energy, bioelectromagnetic therapy, and others, and subsequently described.

A device may release the agent by electromotive administration, also referred to as iontophoresis, using a small electrical current passed through the nerve from the point of agent administration or delivery. In this embodiment, the device contains an electrode, i.e., an anode and/or cathode depending upon the charge state of the agent(s). The device may contain both anode and cathode to accommodate different agents contained in different compartment of the device. An electrode of opposite polarity (cathode and/or anode) is inserted at a site opposite the device. For example, one electrode may be located on a contact lens inserted in the eye, and the other electrode may be positioned at the area of the occipital lobe, the visual processing center of the brain located at the back of the skull.

The flow of current from the point of administration through the nerve is regulated externally by an energy source. When current is applied, an electrical potential difference is generated between the two electrodes, facilitating agent transport through the nerve. Such administration may permit a relatively higher concentration of agent to be delivered diffusively at a site requiring agent. The dose of agent delivered depends upon the current and duration selected. In one embodiment, a current between about 0.5 mA and about 4 mA is applied for between a few seconds to about 20 min. Iontophoresis delivery itself has no side effects and there is no pain associated with agent administration. Thus, it may be used in any embodiment.

For use with ultrasound, a water soluble gel is applied to the skin on and surrounding the area to be treated with ultrasound radiation. The source of ultrasond energy is set at the desired level of intensity, for example, 12.5 W, with the timer set for fifteen minutes. A transducer is gently placed on the prepared area and sonic energy is applied using continual movement of the transducer in either a clockwise or counterclockwise direction, limiting the area to a circle of about 1-1 2 inches in diameter. Consistent pressure is applied over the area.

Bioelectromagnetic energy may be used, for example, using applied pulsed and direct current electromagnetic fields. Application of electromagnetic fields may be used for nerve stimulation; for example, transcutaneous, transcranial, neuromagnetic, electromyography, electroencephalography, electroreinography, and low energy emission therapy.

In one embodiment, the agent is introduced at a sensory site with a high concentration of nerve endings. In another embodiment, the agent may be nasally introduced by a spray or aerosol, without inhalation (i.e., the agent does not reach the lungs) and may access a number of nerve terminals in the nose. Agent absorption at the olfactory region of the nose provides a potential for agent availability to the central nervous system.

Agent absorption is influenced by the residence (contact) time between the agent and the epithelial tissue. Mucociliary clearance is inversely related to the residence time and therefore inversely proportional to the absorption of agents administered. Residence time in the nasal cavity may be prolonged by using bioadhesive polymers, microspheres, chitosan or by increasing the viscosity of the formulation. Formulations for intranasal administration include agents in solutions, suspensions, and/or emulsions administered as drops, sprays, or aerosols, and gels and/or ointments administered by application to the mucosa or squirting into the nose and/or mouth. In one embodiment, the agent is formulated for delivery as a nasal spray that is provided to the nasal mucosa and is not inhaled, that is, it does not reach the lungs. In one embodiment, a spray is formulated so that it is visible upon administration. This may be beneficial in that the individual is reinforced by several sensory aspects, that is, sight (seeing the spray administered), taste for a mouth spray (tasting the spray and having it be a certain flavor in the mouth; aroma for a mouth spray and a nasal spray (smelling the spray and having it be a certain aroma), consistency of the spray (e.g., a fine mist, an aerosol, etc.).

As another example, the agent may be introduced into the Eustachian tube to access the inner ear and/or brain. The Eustachian tube is a membrane-lined tube that connects the middle ear to the back of the nose (“throat” or pharynx). The pharynx extends from the base of the skull to the level of the sixth cervical vertebra. Inferiorly, it opens into the larynx (respiratory system) and esophagus (digestive system). The pharynx is divided into the nasopharynx, oropharynx, and laryngopharynx. The nasopharynx is the portion of the pharynx that is posterior to the nasal cavity and extends inferiorly to the uvula. The oropharynx is the portion of the pharynx that is posterior to the oral cavity. The laryngopharynx is the most inferior portion of the pharynx that extends from the hyoid bone down to the lower margin of the larynx.

Because of anatomy, agent can access the Eustachian tube by administration into either the nose or the pharynx. In one embodiment, agent may be administered into the nose, e.g., formulated as an inhalable, a spray, a topical, etc. as a route from the nose to the Eustachian tube. In another embodiment, agent may be administered into the mouth, e.g., formulated as an inhalable, spray, aerosol etc. for spraying or breathing into the mouth but not entering the lungs, as a route from the pharynx to the Eustachian tube. Agent can then exit the body by simple exhalation through the nose and/or mouth. In these embodiments, agent has access via the Eustachian tube to the middle ear, inner ear, and brain via the eighth cranial nerve (vestibulocochlear nerve).

In one embodiment, agents formulated with or in microspheres provide more prolonged contact with the nasal mucosa and thus enhance absorption. Microspheres for nasal applications have been prepared using biocompatible materials, such as starch, albumin, dextran and gelatin (Bjork E and Edman P., Microspheres as nasal delivery system for peptide drugs. J. Controlled Release 21, 165 (1992), which is expressly incorporated by reference herein).

In embodiments using a type of facilitated transport such as a vector, iontophoretic delivery, etc., the concentration of agent may be lower than in embodiments where such transport is not facilitated, because of directed or facilitated transport that results in a higher concentration of agent reaching the desired site. In another embodiment, a supratherapeutic but non-toxic dose of agent may be administered in an area adjacent the site of administration.

Administration may be intermittent, sustained for a particular duration, as needed, to achieve a desired effect, dose, etc. Multiple administrations of agent may be used. An agent may be formulated to be taken up by the neuron by receptor-mediated endocytosis if the agent is conjugated to a suitable moiety, such as a ligand for a particular receptor. Receptor mediated endocytosis is a process by which cells internalize molecules or viruses. It requires ligand interaction with a specific binding protein, a receptor, on or in the cell membrane. Ligands that are internalized by receptor-mediated endocytosis include, but are not limited to, toxins and lectins such as diphtheria toxin, pseudomonas toxin, cholera toxin, ricin, and concanavalin A; viruses such as Rous sarcoma virus, Semliki forest virus, vesicular stomatitis virus, and adenovirus; serum transport proteins and antibodies such as transferrin, low density lipoprotein, transcobalamin, IgE, polymeric IgA, maternal IgG, and IgG, via Fc receptors; and hormones and growth factors such as insulin, epidermal growth factor, growth hormone, thyroid stimulating hormone, nerve growth factor, calcitonin, glucagon, prolactin, luteinizing hormone, thyroid hormone, platelet derived growth factor, interferon, and catecholamines. An example of agent internalization into a cell by receptor-mediated endocytosis is the conjugation of transferrin with therapeutic drugs, proteins, or genetically by infusion of therapeutic peptides or proteins into the structure of transferrin. Also, conjugation of the agent to the OX26 monoclonal antibody which recognizes the transferrin receptor may be used to deliver therapeutic agents inside the cell via receptor-mediated endocytosis.

Alternatively, the agent may be introduced into the cell by incorporating the agent within liposomes. As known to one skilled in the art, liposomes are vesicles surrounded by a lipid membrane resembling that of a cell and are endocytosed by the cell.

Cigarettes contain 6 to 11 mg of nicotine, of which the smoker typically absorbs 1 to 3 mg, irrespective of the nicotine-yield ratings provided by the tobacco company. The typical pack-per-day smoker absorbs 20 to 40 mg of nicotine each day, achieving plasma concentrations of 25 to 35 mg per milliliter by the afternoon. The plasma half-life of nicotine is approximately two hours. In accordance with the present invention the amount of nicotine in the composition to be and the time frame for titrating the dose depends upon a number of factors such as factors influencing nicotine absorption and subject-dependent factors (i.e., smoking behavior, lung clearance rat, morphological factors, physiological factors, age, sex, weight, frequency of smoking, nicotine tolerance of the smoker, type of delivery vehicle, daily stress patterns, and demographic factors, in part, the amount of nicotine sufficient to satisfy the smoker's craving for nicotine).

In one embodiment, the system and method duplicates such sensory and behavior-related aspects of smoking. Examples are an individual's appreciation, either consciously or not, of aspects such as sense of aroma (smell of a lit or unlit cigarette), taste of a cigarette in the mouth), sight (seeing the cigarette, seeking exhaled smoke), feel (cylindrical shape and weight in the hand, feel between the lips, rituals of hand to mouth coordination and placement), etc.

In doing so, it allows the individual a similar experience with an agent provided in delivery vehicle that mimics a cigarette's shape, style, dimensions, etc. For example, the vehicle may be composed of polymers that, with heating such as occurs with a lit cigarette, results in visible manifestations that mimic smoke from a lit cigarette. The individual thus obtains the agent, disseminated through oral and nasal neural conduits, that permits him/her to reduce dependency, while simultaneously maintaining smoking associated rituals (holding a cylindrical device between fingers and lips, lighting it, seeing smoke from it, etc.). Such polymers are biocompatible and are known to one skilled in the art.

An effective amount of agent, as used herein, is that amount effective to achieve the specified result. That is, it is the amount needed to reduce or inhibit an individual's tobacco-smoking addiction-related behavior, dependency characteristics, incentive/reward effects, and cigarette associated cravings. The amount diminishes or relives one or more symptoms or conditions resulting from cessation or withdrawal of the drug. Thus, the method is not limited to any particular dose. Doses will generally be those delivered in other nicotine delivery devices for smoking cessation, such as transdermal patches.

In one embodiment, an agent may be administered to provide a daily dose ranging from about 15 mg/kg to about 600 mg/kg. In one embodiment, an agent may be administered to provide a daily dose ranging from about 750 μg/kg to about 100 mg/kg. In one embodiment, an agent may be administered to provide a daily dose ranging from about 100 μg/kg to about 1 mg/kg. For a slow release delivery vehicle, the amount of agent released may range, in one embodiment, from about 5 μg per day to about 500 μg per day. In another embodiment using a slow release delivery vehicle, the amount of agent released may range from about 0.1 μg per day to about 10 μg per day. The length of time these doses are administered or the number of administration cycles with decreasing amounts of the agent will vary depending on an individual's response.

The system and method has applicability to smokers wishing to quit or trying to quit who have experienced all or any of the nicotine withdrawal symptoms associated with smoking cessation. Symptoms include nicotine craving, irritability, frustration, anger, anxiety, drowsiness, sleep disturbances. Impaired concentration, nervousness, restlessness, decreased heart rate, increased appetite and weight gain, and others.

In one embodiment, the disclosed method and system may be used to provide neural conduit dissemination for other disorders that are related to dopamine reuptake. The agent can be an anti-attention-deficit-disorder agent such as, but not limited to, methylphenidate; dextroamphetamine; tricyclic antidepressants such as imipramine, desipramine, and nortriptyline; psychostimulants such as pemoline and deanol, etc. The agent can be a non-tobacco anti-addictive-disorder agent such as, but not limited to, tricyclic antidepressants; monoamine oxidase (MAO) inhibitors; glutamate antagonists such as ketamine HCl, dextromethorphan, dextrorphan tartrate and dizocilpine (MK801); degrading enzymes such as anesthetics and asparatate antagonists; gamma aminobutyric acid (GABA) agonists such as baclofen and muscimol HBr; reuptake blockers; degrading enzyme blockers; glutamate agonists such as D-cycloserine, carboxyphenylglycine, L-glutamic acid, and cis-piperidine-2,3-dicarboxylic acid; aspartate agonists; GABA antagonists such as gabazine (SR-95531), saclofen, bicuculline, picrotoxin, and (+) apomorphine HCl; and dopamine antagonists such as spiperone HCl, haloperidol, and (−) sulpiride. The agent can be an anti-opiate agent such as, but not limited to, methadone, clonidine, iofexidine, levomethadyl acetate HCl, naltrexone, and buprenorphine. The agent can be an anti-cocaine agent such as, but not limited to, desipramine, amantadine, fluoxidine, and buprenorphine. The agent can be an appetite suppressant such as, but not limited to: fenfluramine, phenylpropanolamine, and maxindol. The agent can be an anti-lysergic acid diethylamide (anti-LSD) agent such as, but not limited to, diazepam. The agent can be an anti-phencyclidine (anti-FCP) agent such as, but not limited to, haloperidol. The agent can be an anti-Parkinson's-disease agent such as, but not limited to, dopamine precursors, such as levodopa, L-phenylalanine, and L-tyrosine; neuroprotective agents; dopamine agonists; dopamine reuptake inhibitors; anticholinergics such as amantadine and memantine; and 1,3,5-trisubstituted adamantanes, such as 1-amino-3,5-dimethyl-adamantane as disclosed in U.S. Pat. No. 4,122,193 to Sherm et al. The agent can be an anti-depression agent such as, but not limited to, amitriptyline, clomipramine, doxepine, imipramine, trimipramine, amoxapine, desipramine, maprotiline, nortriptyline, protripyline, fluoxetine, fluvoxamine, paraxetine, setraline, venlafaxine, bupropion, nefazodone, trazodone, phenelzine, tranylcypromine and selegiline. The ager I can be an anxiolytic agent such as, but not limited to, benzodiazepines such as alprazolam, chlordiazepoxide, clonazepam, clorazepate, diazepam, halazepam, lorazepam, oxazepam, and prazepam; non-benzodiazepine agents such as buspirone; and tranquilizers such as barbituates. The agent can be an antipsychotic drug such as, but not limited to, phenothiazines such as chlorpromazine, mesoridazine besylate, thioridazine, acetophenazine maleate, fluphenazine, perphenazine, and trifluoperazine; thioxanthenes such as chlorprothixene, and thiothixene; and other hetercyclic compounds, such as clozapine, haloperidol, loxapine, molindone, pimozide, and risperidone. Anti-psycotic drugs also include chlorpromazine HCl, thioridazine HCl, fluphenazine HCl, thiothixene HCl, and molindone HCl. The agent can be an anti-obesity drug such as, but not limited to, alpha-adrenergic receptor agonists, alpha-3 receptor agonists such as, but not limited to, fenfluramine; dexfenfluramine; sibutramine; bupropion; fluoxetine; phentermine; amphetamine; methamphetamine; dextroamphetamine; benzphetamine; phendimetrazine; diethylpropion; mazindol; phenylpropanolamine; norepinephrine-serotonin reuptake inhibitors such as sibutramine; and pancreatic lipase inhibitors such as orlistat.

In one embodiment a primary agent, including a pharmaceutically acceptable salt, analog, or derivative as previously disclosed, collectively termed agent, is used in combination with at least one other second agent. Any of the above listed compounds may be a primary or secondary agent. The primary and secondary agent can act additively or synergistically. In one embodiment, an agent is administered concurrently with a second agent, and either as part of the same composition or in a different composition. The second agent can be useful for treating, reducing symptoms of, etc. a second malady resulting from the disorder for which the primary agent is administered. As only one non-limiting example, nicotine may be a primary agent administered for smoking cessation, and an appetite suppressant may be a secondary agent administered for reducing hunger that comes with nicotine withdrawal. Other examples will be appreciated by one skilled in the art.

In the embodiment where nicotine is the agent and is administered for smoking cessation, its duration of administration is limited and is relatively short-term, on the order of weeks to a few months. In one embodiment, nicotine is administered for one to two weeks. In one embodiment, nicotine is administered for about one month. In one embodiment, nicotine is administered for about two months. In one embodiment, nicotine is administered for about three months. In one embodiment, the dose of nicotine is titrated downward, either by the individual or by a medical practioner, so that the amount of nicotine is gradually reduced to the point where agent may be minimized or eliminated. It will be appreciated that the same delivery vehicle may be used without agent, or with a non-pharmaceutical compound such as herbs or other aroma/taste imparting components, for longer durations. In this embodiment, duration may be many months (e.g., six months), a year, or for more extended periods including indefinitely.

From the above description, other variations or embodiments of the method and system will also be apparent to one of ordinary skill in the art. As one example, an individual may select components to achieve a desired aroma and/or taste (e.g., menthol or lack of menthol flavor). Thus, the forgoing embodiments are not to be construed as limiting the scope of this invention.

What is claimed is:

1. A method of disseminating a biocompatible agent to an individual, the method comprising
providing to an individual an agent capable of exerting an affect at a distal neural site, the agent provided by a non-optical ocular route of administration for dissemination via a neural conduit to exert the effect at the distal neural site, wherein the distal neural site is a cholinergic receptor.

2. A method of disseminating a biocompatible agent to a patient to treat nicotine addiction, the method comprising
providing to a patient in need thereof at a first peripheral nervous system site, an agent capable of exerting via dissemination by a neural conduit an effect at at least one of a second peripheral nervous system site or a central nervous system site, the agent administered at at least one acupuncture site by at least one of injection, transdermal administration, or facilitated topical administration wherein the peripheral nervous system site or a central nervous system site is a cholinergic receptor.

3. A method of disseminating a biocompatible agent to a patient, the method comprising
providing to at least one of an oral cavity or a nasal cavity of a patient for dissemination via a neural conduit from a Eustachian tube to at least one of a second peripheral nervous system site or a central nervous system site, an agent capable of exerting an effect at the site.

4. The method of claim 1 wherein the distal neural site is a central nervous system site or a non-ocular peripheral nervous system site.

5. The method of claim 1 wherein the route of administration is at least one of intraocular injection or intraocular implantation.

6. The method of claim 1 wherein the agent is disseminated in the perineurium of the optic nerve.

7. The method of any of claim 1, claim 2, or claim 3 wherein dissemination is facilitated by application of at least one of electrical current, ultrasound energy, radiant energy, bioelectromagnetic therapy, or thermal energy.

8. The method of any of claim 1, claim 2, or claim 3 wherein the agent is at least one of a drug, a vaccine, a peptide, a protein, or a vector containing a gene therapy agent.

9. The method of any of claim 1, claim 2, or claim 3 wherein the agent is conjugated to a transport facilitating moiety.

10. The method of any of claim 1, claim 2, or claim 3 wherein the agent is formulated for controlled release.

11. The method of any of claim 1, claim 2, or claim 3 wherein the agent is selected from at least one of a macrolide, anti-prostaglandin, matrix metalloproteinase inhibitor, anti-viral agent, antioxidant, anti-cell migration agent, angiogenic agent, anti-angiogenic agent, or anti-neoplastic agent.

12. The method of any of claim 1, claim 2, or claim 3 wherein the agent is for alleviation of age related macular degeneration.

13. The method of any of claim 1, claim 2, or claim 3 wherein the agent is formulated as at least one of a solution, suspension, emulsion, microspheres, manospheres, lipsomes, microparticles, or nanoparticles.

14. A method of disseminating a biocompatible agent, the method comprising
providing to an individual at a first neural site an agent selected from the group consisting of an acetylcholinesterase inhibitor, an L-type calcium channel modulator, an agonist of a nicotinic α-7 receptor, an inhibitor of phosphodiesterase 10, an inhibitor of phosphodiesterase 4, and combinations thereof, the agent disseminated along a neural conduit to a central nervous system site in need of therapy, the agent administered by at least one of
a non-topical ocular route,
injection, transdermal application, or facilitated topical administration of at least one acupunture site,
administration at an olfactory site, or
pharynx or nasal administration to a Eustachian tube.

15. The method of claim 14 wherein the agent is targeted to a region of the brain.

16. The method of claim 14 wherein administration is facilitated by application of at least one of electrical current, ultrasound energy, radiant energy, bioelectromagnetic therapy, or thermal energy.

17. A method to reduce an individual's nicotine craving, the method comprising
disseminating nicotine through a neural conduit by administering nicotine to a peripheral nervous system site increasing doses over time, such that nicotine is transported substantially by the neural conduit and substantially free of a vascular conduit, to at least one cholinergic receptor in the central nervous system, thus decreasing at least one of the nicotine amount or the duration of nicotine administration by use of the neural conduit.

18. The method of claim 17 wherein the peripheral nervous system site is selected from the group consisting of nose, mouth, skin acupunture site, and combinations thereof.

19. The method of claim 17 wherein nicotine is administered by inhalation.

20. The method of claim 17 wherein nicotine is formulated as a spray and is administered to at least one of a nasal cavity or an oral cavity of the individual.

21. The method of claim 17 wherein nicotine is formulated with polymers which, upon heating, are visible as a smoke-mimetic.

22. The method of claim 17 wherein administration is facilitated.

23. The method of claim 17 further comprising a transport facilitating moiety.

24. The method of claim 17 wherein nicotine is administered as particles ranging from about 25 nm to about 200 nm.

25. The method of claim 17 wherein nicotine is in a controlled release formulation.
